(19) United States
(12) Patent Application Publication
(10) Pub. No.: US 2016/0262898 A1
(43) Pub. Date: Sep. 15, 2016

(54) TIBIAL IMPLANT FOR USE IN KNEE ARTHROPLASTY

(71) Applicants: Nolan C. Jones, Warsaw, IN (US); James C. Harris, Warsaw, IN (US)

(72) Inventors: Nolan C. Jones, Warsaw, IN (US); James C. Harris, Warsaw, IN (US)

(21) Appl. No.: 15/065,008

(22) Filed: Mar. 9, 2016

Related U.S. Application Data
(60) Provisional application No. 62/132,127, filed on Mar. 12, 2015.

(51) Int. Cl. A61F 2/38

(52) U.S. Cl. CPC A61F 2/389; A61F 2002/30708

(57) ABSTRACT

Various apparatuses and related methods are disclosed. According to one example, a tibial implant is disclosed that can be configured for attachment to a tibia in a knee arthroplasty. The tibial implant can include a baseplate having a first portion and a second portion, the first portion is symmetrically shaped, sized, and positioned with respect to the second portion and one or more fixation features configured to extend from a distal surface of the baseplate and configured for attachment to a resected proximal surface of the tibia.

### CLAIM OF PRIORITY

This application claims the benefit of U.S. Provisional Patent Application Ser. No. 62/132,127, filed on Mar. 12, 2015, the benefit of priority of which is claimed hereby, and which is incorporated by reference herein in its entirety.

### TECHNICAL FIELD

The present application relates to orthopedic prostheses, and more particularly, to a tibial implant for use in a knee arthroplasty procedure.

### BACKGROUND

Orthopedic procedures are commonly utilized to repair and/or replace damaged bone and tissue in the human body. For example, a knee prosthesis may be used to restore natural knee function by repairing damaged or diseased articular surfaces of the femur and/or tibia. Knee prostheses may include a femoral component implanted on the distal end of the femur, which articulates with a tibial component implanted on the proximal end of the tibia to replicate the function of a healthy natural knee.

Tibial implants (trays) are utilized in various knee arthroplasty procedures such as in unicompartmental, bicompartmental, total (TKA), and total sparing ACL/PCL procedures. Typically, a tibial implant can have a side secured to the bone stock of a resected proximal tibia. A bearing component can be secured to an opposing side of the tibial implant. The tibial implant allows the bearing surface to be fixed so as to replicate the anatomical articulation of a knee joint when used in combination with a femoral prosthesis.

### OVERVIEW

Traditional tibial implants can be specifically adapted for use in a particular compartment of the knee such as the left tibia medial compartment or left tibia lateral compartment. Some traditional tibial implants have been developed for unicompartmental knee arthroplasty procedures that can be used in two compartments such as the left tibia medial compartment and the right tibia lateral compartment. The present inventors have recognized that a symmetric anterior/posterior tibial implant can reduce the overall number of tibial implant designs needed for various knee arthroplasty procedures such as in unicompartmental, bicompartmental, TKA, and TKA sparing ACL/PCL, as a single implant design can be used in various knee compartments (e.g., lateral and medial) for both the left and right tibia. The reduction in tibial implant designs that result from the disclosed symmetric tibial implant design can result in reduced manufacturing and inventory costs and reduce the number of selection decisions made by the surgeon or other personnel.

The present inventors further recognize that the use of three or more fixation features such as pegs for a unicompartmental tibial implant can create installation challenges. In particular, it can be challenging for a physician to drill three or more distinct holes in a single compartment at the proximal end of the tibia. Therefore, a modular system of fixation features such as pegs or other components is disclosed. As will be discussed in further detail subsequently with reference to the following examples, at least two fixation features can be attached to and extend from a distal surface of a baseplate of the tibial implant and can be configured for attachment to a resected proximal surface of the tibia. In some examples, the at least two fixation features can include a first integrally formed component generally centrally disposed between a first portion and a second portion of the tibial implant, and a second removable component that can be attached to one of two apertures formed in the baseplate. According to some examples, the first of the two apertures can be formed in the first portion and the other of the two apertures can be formed in the second portion. In further examples, the removable component can be attached to the more anteriorly disposed of the two apertures.

To further illustrate the components and methods disclosed herein, a non-limiting list of examples is provided here:

In Example 1, a tibial implant configured for attachment to a tibia in a knee arthroplasty, the tibial implant can comprise a baseplate and one or more fixation features. The baseplate can have a first portion and a second portion. The first portion can be substantially symmetrically shaped, sized, and positioned with respect to the second portion. The one or more fixation features can be configured to extend from a distal surface of the baseplate and can be configured for attachment to a resected proximal surface of the tibia.

In Example 2, the tibial implant of Example 1, wherein the baseplate can be adapted to be disposed in a single compartment of the tibia and the tibia can comprise either a right tibia or a left tibia.

In Example 3, the tibial implant of Example 2, wherein the single compartment can comprise a left tibia medial compartment, aloft tibia lateral compartment, a right tibia medial compartment, or a right tibia lateral compartment.

In Example 4, the tibial implant of Example 3, wherein the first portion can be more anteriorly disposed with respect to the second portion in the left medial compartment and the first portion can be more posteriorly disposed with respect to the second portion in the left lateral compartment.

In Example 5, the tibial implant of any one or any combination of Examples 1 to 4, wherein the first portion comprises one half of the baseplate and the second portion comprises a second half of the baseplate.

In Example 6, the tibial implant of any one or any combination of Example 1 to 5, wherein the one or more fixation features can comprise a single integrally formed component generally centrally disposed between the first portion and the second portion, and a removable component configured to be attached to one of at least two apertures formed in the baseplate, wherein a first one of the apertures is formed in the first portion and a second one of the apertures is formed in the second portion.

In Example 7, the tibial implant of Example 6, wherein the removable component can be attached to a more anteriorly disposed of the at least two apertures.

In Example 8, the tibial implant of any one or any combination of Examples 1 to 7, wherein the baseplate can have a first groove along an edge of the first portion and a matching second groove along an edge of the second portion.

In Example 9, the tibial implant of any one or any combination of Examples 1 to 8, wherein the tibial implant can include a porous structure.

In Example 10, the tibial implant of Example 9, wherein the porous structure can include tantalum.

In Example 11, the tibial implant of any one or any combination of Examples 9 or 10, wherein the porous structure can comprise at least one of the distal surface of the baseplate and the one or more fixation features.

In Example 12, the tibial implant of any one or any combination of Examples 1 to 11, wherein the knee arthroplasty can comprise a bicompartmental knee arthroplasty and the tibial implant can comprise two tibial implants as recited in Example 1, a first of the two tibial implants can be configured to be disposed in a medial compartment of the tibia and a second of the two tibial implants can be configured to be disposed in a lateral compartment of the same tibia.

In Example 13, a unicompartmental tibial component can comprise a baseplate and one or more fixation features. The baseplate can have a first portion and a second portion. The first portion can be symmetrically shaped and sized with respect to the second portion such that the tibial component is configured to be disposed in a left tibia medial compartment, a left tibia lateral compartment, a right tibia medial compartment, or a right tibia lateral compartment. The one or more fixation features can be configured to be attached to and extend from a distal surface of the baseplate and configured for attachment to a resected proximal surface of a tibia.

In Example 14, the tibial component of Example 13, wherein an orientation of the baseplate can be reversible such that a posterior edge of the baseplate becomes an anterior edge of the baseplate.

In Example 15, the tibial component of Example 14, wherein the one or more fixation features can comprise a single integrally formed peg generally centrally disposed between the first portion and the second portion, and a removable peg configured to be attached to one of at least two apertures formed in the baseplate, wherein a first one of the apertures is formed in the first portion and a second one of the apertures is formed in the second portion.

In Example 16, the tibial component of Example 15, wherein the removable peg can be attached to the anteriorly disposed of the at least two apertures.

In Example 17, a method of performing a knee arthroplasty, the method can comprise resecting a proximal surface of a tibia, and attaching a symmetric tibial implant configured to be disposed in a left tibia medial compartment, a left tibia lateral compartment, a right tibia medial compartment, or a right tibia lateral compartment to the resected proximal surface of the tibia.

In Example 18, the method of Example 17, can comprise attaching a fixation component to one of at least two apertures formed in a baseplate of the tibial implant, wherein a first one of the apertures is formed in a posterior portion of the baseplate and a second one of the apertures is formed in an anterior portion of the baseplate.

In Example 19, the method of any one or any combination of Examples 17 to 18 wherein resecting a proximal surface of the tibia can comprise resecting one or both of the medial and lateral articular surfaces of the tibia.

In Example 20, the method of any one or any combination of Examples 17 to 19 wherein resecting a proximal surface of the tibia can comprise resecting one or both of the medial and lateral articular surfaces of the left tibia, and resecting one or both of the medial and lateral articular surfaces of the right tibia,

In Example 21, the system or method of any one or any combination of Examples 1-20 can optionally be configured such that all elements or options recited are available to use or select from.

These and other examples and features of the present systems and methods will be set forth in part in the following Detailed Description. This Overview is intended to provide non-limiting examples of the present subject matter it is not intended to provide an exclusive or exhaustive explanation. The Detailed Description below is included to provide further information about the present tibial prosthesis systems and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is an anterior view of a natural femur and tibia.

FIG. 1B is an elevated view of the tibia of FIG. 1A.

FIG. 1C is a posterior view of the tibia of FIGS. 1A mod 1B, with the anatomical features shown in FIG. 1B removed.

FIG. 2 is an elevated perspective view of a tibial implant in accordance with an example the present application.

FIG. 2A is a view of a top portion of the tibial implant of FIG. 2.

FIG. 2B is a view of a first side of tibial implant of FIG. 2.

FIG. 2C is a view of a second side of the tibial implant of FIG. 2.

FIG. 3 is a view of an underside of the tibial implant of FIG. 2.

FIG. 4 is a perspective view of the underside of the tibial implant of FIG. 2 and additionally including a removable peg.

### DETAILED DESCRIPTION

The present application relates to devices and methods for a tibial prosthesis that can be used various knee arthroplasty procedures including a unicompartmental knee replacement procedure. The disclosed tibial prosthesis can include two substantially symmetric portions e.g., an anterior portion and a posterior portion). According to one example, the tibial prosthesis can be adapted to be disposed in a single compartment of the tibia. This single compartment can comprise a left tibia medial compartment, a left tibia lateral compartment, a right tibia medial compartment, or a right tibia lateral compartment. Thus, a single tibial prosthesis configuration can be utilized in all the aforementioned compartments for either the left or fight tibia. In some examples, the positioning of the tibial prosthesis can be reversed (an anterior portion becoming the posterior portion) to facilitate installation. Such could be the case if the disclosed tibial prosthesis configuration is used for both the lateral and medial compartments of the right tibia or the left tibia.

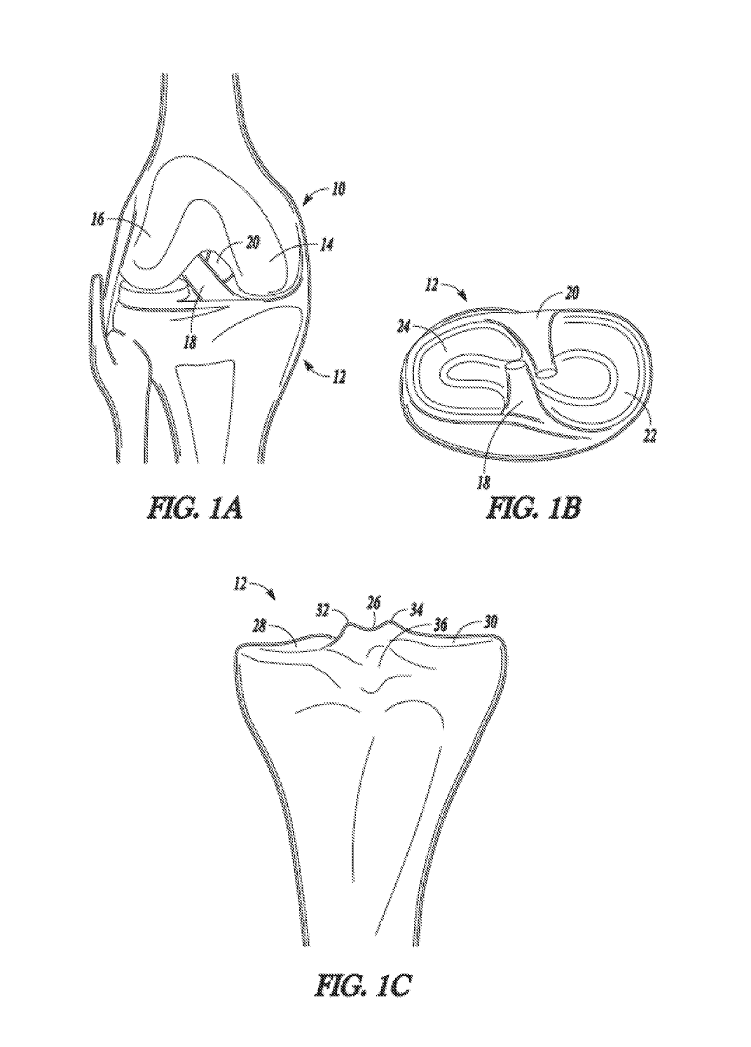

FIG. 1A illustrates a natural femur 10 and tibia 12. The femur 10 can include medial 14 and lateral 16 condyles at a distal end of the femur 10. Various ligaments can be attached to the femur 10 and/or the tibia 12. An anterior cruciate ligament (ACL) 18 can extend from an anterior side of the tibia 12 to the femur 10, and a posterior cruciate ligament (PCL) 20 can extend from a posterior side of the tibia 12 to the femur 10. FIG. 1B is a top view of the tibia 12 and further illustrates some of these ligaments as well as a medial meniscus 22 and a lateral meniscus 24 that are located between the tibia 12 and the medial 14 and lateral 16 condyles.

FIG. 1C illustrates a posterior side view of the tibia 12 with the ligaments and other components shown in FIG. 1B removed. The tibia 12 can include an intercondylar eminence 26, which is a bony elevation or raised area between a medial articular surface 28 and a lateral articular surface 30 at a proximal end of the tibia 12. The intercondylar eminence 26 can include medial 32 and lateral 34 tubercles extending from the intercondylar eminence 26. The ACL 18 and PCL 20 are attached to the tibia 12 at locations anterior and posterior, respectively, to the intercondylar eminence 26. For reference, the PCL 20 is attached to the tibia 12 at a location 36 on a posterior end of the tibia 12.

In a unicompartmental knee arthroplasty procedure, portions of the distal end of the femur 10 and the proximal end of the tibia 12 are resected and replaced with a femoral prosthesis and a tibial prosthesis. As part of performing the resections to the tibia 12, cuts are performed on the affected condyle only, and the unaffected side of the tibia 12 is not disturbed. Once the tibial cut has been performed, a femoral cut is made. The size of the cuts can be based upon how much of the tibia and/or femur must be removed due to disease, plus whatever additional spacing is required to accommodate the closest standard sized tibial prosthesis. The tibial cuts remove a portion of the tibia generally interfacing with the femoral prosthesis creating a compartment. After the tibial cut is made, the tibial prosthesis is temporarily implanted in the compartment and the alignment of the leg is analyzed. To adjust the leg alignment, the practitioner can replace the tibial prosthesis with one of a different thickness, can remove more bone, or can do both. This procedure is followed until the leg is in the desired alignment and balance. After which, a permanent tibial prosthesis can be implanted.

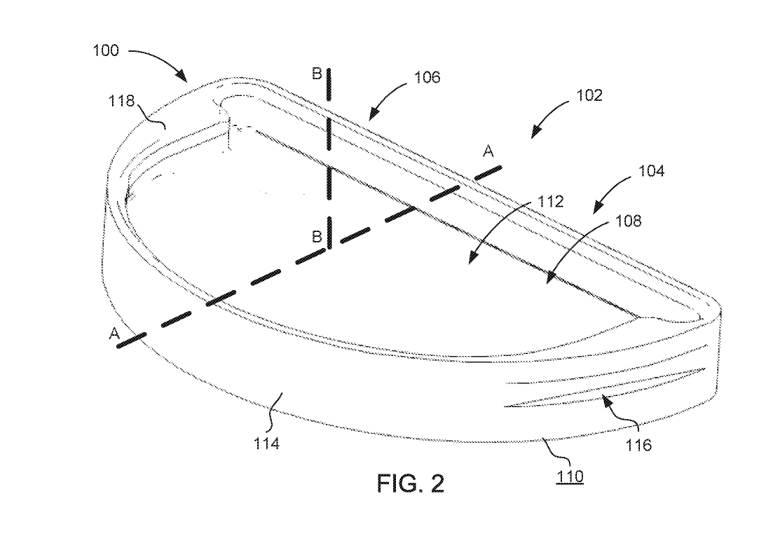

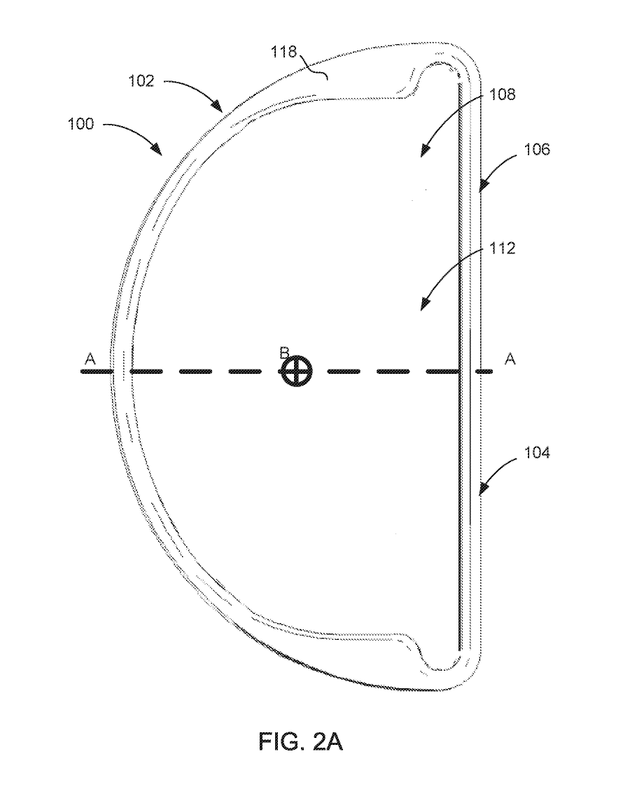

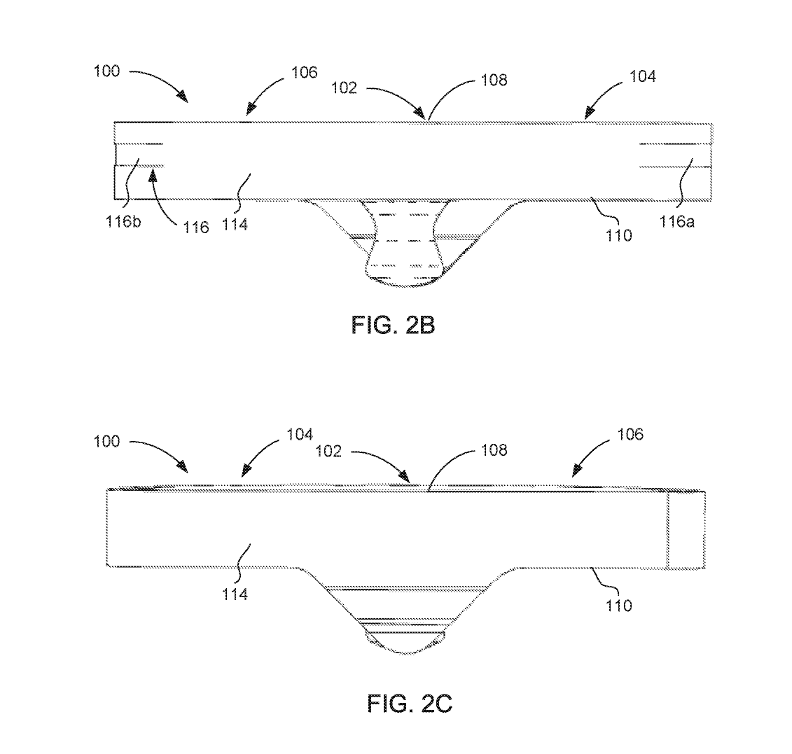

FIGS. 2-2C show an example of a tibial implant 100 used as a baseplate in unicompartmental knee arthroplasty a procedure. The tibial implant 100 can be placed on a resected surface of a proximal tibia and can be configured to engage with another component of the knee prosthesis, such as a tibial bearing component or insert (not shown). The tibial implant 100 can be symmetrically shaped as indicated by axis A-A and can include a baseplate 102 with a first portion 104 that is symmetric with a second portion 106. According to the illustrated example of FIG. 2, the first portion 104 can comprise one half of the baseplate 102 and the second portion 106 can comprise a second half of the baseplate 102. Additionally, the baseplate 102 can include a proximal surface 108, a distal surface 110, a recess 112, side surface 114, and grooves 116 (only one is illustrated in FIG. 2).

The tibial implant 100 can be adapted to be disposed in a single compartment of the tibia and the tibia can comprise either a right tibia or a left tibia. More particularly, the symmetric design of the tibial implant 100 (with the first portion 104 symmetrically shaped, sized, and relatively located with respect to the second portion 106) can allow for implantation of the tibial implant 100 in a left tibia medial compartment, a left tibia lateral compartment, a right tibia medial compartment, or a right tibia lateral compartment. Thus, according to some examples, multiple tibial implants such as tibial implant 100 can be utilized within a patient simultaneously (e.g., one tibial implant 100 can be utilized for the left tibia lateral compartment and another tibial implant 100 can be utilized for the right tibia medial compartment). For example, if the knee arthroplasty comprises a bicompartmental knee arthroplasty, the tibial implant 100 can comprise two similarly shaped but differently sized tibial implants, a first of the two tibial implants can be configured to be disposed in a medial compartment of the tibia and a second of the two tibial implants can be configured to be disposed in a lateral compartment of the same tibia. According to further examples, the tibial implant 100 can comprise two indentical (similarly sized and shaped) tibial implants.

According to the example of FIGS. 2 and 2A, the first portion 104 can be symmetrically shaped with respect to the second portion 106. This configuration allows for rotation of the tibial implant 100 about axis B-B according to the implant compartment and knee desired. The orientation of the baseplate 102 can be reversible such that a posterior edge of the baseplate 102 becomes an anterior edge of the baseplate 102 upon rotation. Thus, according to some examples, the first portion 104 can be more anteriorly disposed with respect to the second portion 106 in some implantation dispositions (e.g., upon implantation in the left tibia medial compartment or the right tibial lateral compartment) and can be more posteriorly disposed with respect to the second portion 106 in other implantation dispositions (e.g., upon implantation in the left tibia lateral compartment or the right tibial medial compartment). The symmetric shape of the baseplate 102 can simplify component selection for the surgeon and can additionally reduce manufacturing and inventory complexity.

A size and dimensions of a particular baseplate 102 can be determined based, in part, on the patient's anatomy. The proximal surface 108 of baseplate 102 can be adapted to form an outer rim 118 (FIGS. 2 and 2A) as well as the recess 112. The distal surface 110 (FIG. 2) can be spaced from the proximal surface and can be adapted to couple with one or more fixation features (discussed subsequently). According to some examples, the distal surface 110 of the baseplate 102 can itself comprise one or more fixation features.

Side surface 114 is illustrated in FIGS. 2, 2B, and 2C can extend between the proximal surface 108 and the distal surface 110 and can be adapted to interface with physiology of the tibia (e.g., the intercondylar eminence). The grooves 116 (only one is shown in FIG. 2 but both are illustrated in FIG. 2B) can be symmetric with respect to one another (i.e. can have a same size, shape, and relative location) and can be formed in the side surface 114 at opposing anterior/posterior ends thereof. Thus, one groove 116a (FIG. 2B) can be formed in the first portion 104 and the second groove 116b (FIG. 2B) can be formed in the second portion 106. Therefore, the baseplate 102 can have the first groove 116a along an edge of the first portion 104 and a matching second groove 116b along an edge of the second portion 106. Recess 116 can be adapted to receive a bearing component (not shown) therein. Such bearing component can comprise, for example, a biocompatible polymeric material (e.g., ultra high weight polyethylene) or vitamin E reinforced biocompatible polymeric material as is described in U.S. Pat. Nos. 7,846,376, 8,858,643, 8,771,369, 8,690,954, the entire disclosures of which are incorporated herein by reference. The grooves 116 are adapted to receive one or more surgical instruments, which are used to insert, remove, and/or adjust the tibial implant 100 within the patient.

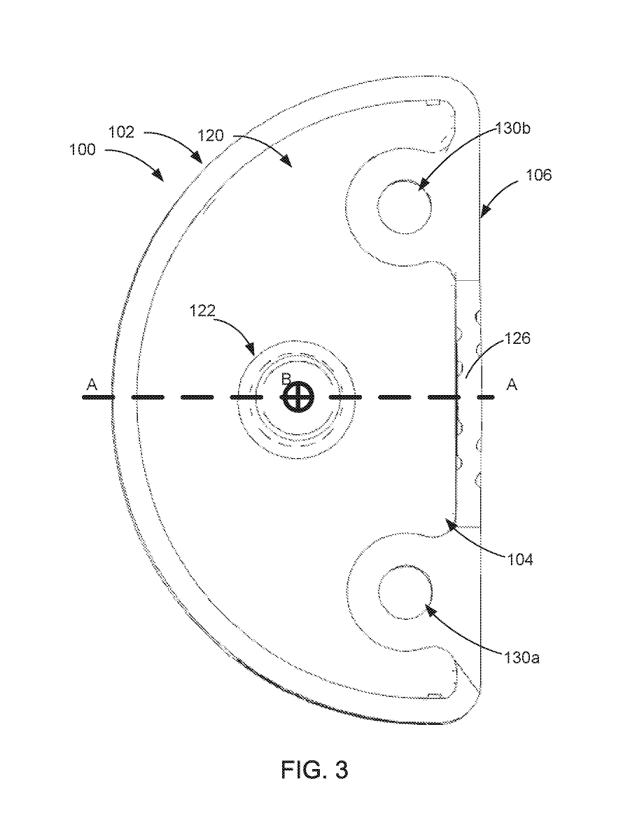

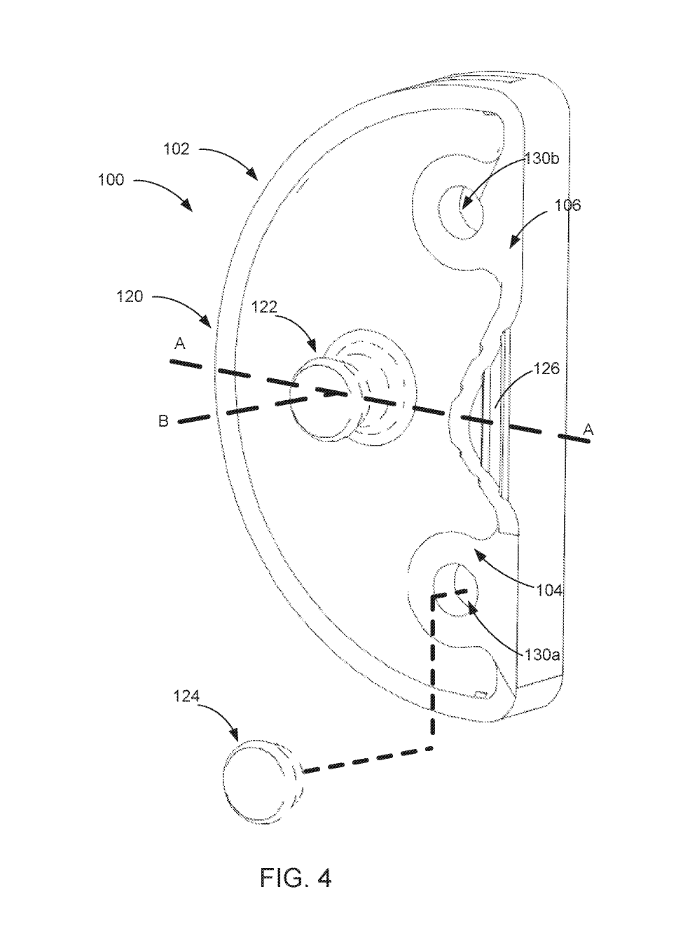

FIGS. 3 and 4 illustrate the distal surface 110 and one or more fixation features 120 of the tibial implant 100 according to an example the present application. According to the illustrated example, the one or more fixation features 120 can include an integrally formed component 122, at least one removable component 124 (FIG. 4), and a keel 126.

According to the example of FIGS. 3 and 4, the one or more fixation features 120 can be attached to and can extend from the distal surface 110 of the baseplate 102. The fixation features 120 can be configured for attachment to a resected proximal surface of the tibia. In one example, the integrally formed component 122 can comprise a single component generally centrally disposed between the first portion 104 and the second portion 106 (e.g., is disposed so as to be bisected by axis A-A). Component 122 can be formed with other portions of the baseplate 102 and can comprise a similar material thereto. According to the illustrated example, the integrally formed component 122 can comprise an hourglass shaped peg. However, in other examples component 122 can comprise another fixation feature such as a bone screw, keel, etc.

As shown in FIG. 4, the at least one removable component 124 can comprise an hourglass shaped peg configured to be attached to one of two apertures 130a and 130b formed in the baseplate 102. However, the at least one removable component 124 can comprise another fixation feature such as a bone screw, keel, porous material, etc. Such attachment can be accomplished by using known attachment techniques, including, but not limited to, bone cement or other adhesion materials, bonding, or mechanical fixation, such as, for example, thread, a snap fit, and other mechanical features to promote fixation. As shown in both FIGS. 3 and 4, the first aperture 130a of the two apertures 130a and 130b can be formed in the first portion 104 and the other aperture 130b of the two apertures 130a and 130b can be formed in the second portion 106. According to some examples, the removable component 124 can be attached to a more anteriorly disposed of the two apertures 130a and 130b. Thus, the modular system of fixation features disclosed (e.g. the removable component 124 attached to a more anteriorly disposed of the two apertures 130a and 130b) can allow the surgeon to drill two holes in the in a single compartment at the proximal end of the tibia. This arrangement can provide the surgeon more space to work within the compartment in which to anchor the tibial implant 100.

According to the example of FIGS. 3 and 4, the keel 126 can extend from the first portion 104 to the second portion 106 of the baseplate 102. In the example as shown in FIGS. 3 and 4, the keel 126 can terminate prior to the anterior and posterior ends of the baseplate 102. In other examples, the keel 126 can extend to one or both of the ends. According to further examples, the keel 126 can comprise several keels disposed in multiple locations along the baseplate 102. It is recognized that various other configurations for the one or more fixation features 120 are contemplated and can be used, but are not specifically discussed herein.

According to some examples, portions of the tibial implant 100 can be formed of different materials. For example, the baseplate 102 can be formed of a biocompatible metal or metal alloy, such as, for example, titanium, a titanium alloy, cobalt chromium, cobalt chromium molybdenum, tantalum, or a tantalum alloy. In an example, the baseplate 102 comprises a Ti-6Al-4V ELI alloy, such as Tivanium® which is available from Zimmer, Inc., of Warsaw, Ind. Tivanium® is a registered trademark of Zimmer, Inc. In another example, all or a portion of the baseplate 102 can be formed of a polymeric material, such as, for example, polyethylene. For example, the baseplate 102 and bearing surface (not shown) can comprise a polymeric material while other portions such as the one or more fixation features 122 can comprise a porous material. Thus, in some examples the one or more fixation features 122 can he formed of a metal or metal alloy. In yet a further example, a porous structure such as a porous metal described below, can be utilized to facilitate bone ingrowth or regrowth. Such porous structure can comprise at least one of the distal surface 110 of the baseplate 102 and the one or more fixation features 122 such as a porous metal described below, to facilitate bone ingrowth or regrowth.

A highly porous metal structure can incorporate one or more of a variety of biocompatible metals. Such structures are particularly suited for contacting bone and soft tissue, and in this regard, can be useful as a bone substitute and as cell and tissue receptive material, for example, by allowing tissue to grow into the porous structure over time to enhance fixation (i.e., osseointegration) between the structure and surrounding bodily structures. According to certain examples of the present disclosure, an open porous metal structure may have a porosity as low as 55%, 65%, or 75% or as high as 80%, 85%, or 90%, or within any range defined between any pair of the foregoing values. An example of an open porous metal structure is produced using Trabecular Metal™ Technology available from Zimmer, Inc., of Warsaw, Ind. Trabecular Metal™ is a trademark of Zimmer, Inc. Such a material may he formed from a reticulated vitreous carbon foam substrate which is infiltrated and coated with a biocompatible metal, such as tantalum, by a chemical vapor deposition (“CVD”) process in the manner disclosed in detail in U.S. Pat. No. 5,282,861 and in Levine, B. R., et al., “Experimental and Clinical Performance of Porous Tantalum in Orthopedic Surgery”, Biomaterials 27 (2006) 4671-4681, the disclosures of which are expressly incorporated herein by reference. In addition to tantalum, other biocompatible metals may also be used in the formation of a highly porous metal structure such as titanium, a titanium alloy, cobalt chromium, cobalt chromium molybdenum, tantalum, a tantalum alloy, niobium, or alloys of tantalum and niobium with one another or with other metals. It is also within the scope of the present disclosure for a porous metal structure to be in the form of a fiber metal pad or a sintered metal layer, such as a Cancellous-Structured Titanium™ (CSTi™) layer. CSTi™ porous layers are manufactured by Zimmer, Inc., of Warsaw, Ind. Cancellous-Structured Titanium™ and CSTi™ are trademarks of Zimmer, Inc.

Generally, a highly porous metal structure will include a large plurality of metallic ligaments defining open voids (i.e., pores) or channels therebetween. The open spaces between the ligaments form a matrix of continuous channels having few or no dead ends, such that growth of soft tissue and/or bone through open porous metal is substantially uninhibited. Thus, the open porous metal may provide a lightweight, strong porous structure which is substantially uniform and consistent in composition, and provides a matrix (e.g., closely resembling the structure of natural cancellous bone) into which soft tissue and bone may grow to provide fixation of the implant to surrounding bodily structures. According to some aspects of the present disclosure, exterior surfaces of an open porous metal structure can feature terminating ends of the above-described ligaments. Such terminating ends can be referred to as struts, and they can generate a high coefficient of friction along an exposed porous metal surface. Such features can impart an enhanced affixation ability to an exposed porous metal surface for adhering to bone and soft tissue. Also, when such highly porous metal structures are coupled to an underlying substrate, a small percentage of the substrate may be in direct contact with the ligaments of the highly porous structure, for example, approximately 15%, 20%, or 25%, of the surface area of the substrate may be in direct contact with the ligaments of the highly porous structure.

An open porous metal structure may also be fabricated such that it comprises a variety of densities in order to selectively tailor the structure for particular orthopedic applications. In particular, as discussed in the above-incorporated U.S. Pat. No. 5,282,861, an open porous metal structure may be fabricated to virtually any desired density, porosity, and pore size (e.g., pore diameter), and can thus be matched with the surrounding natural tissue in order to provide an improved matrix for tissue ingrowth and mineralization. According to certain embodiments, an open porous metal structure may be fabricated to have a substantially uniform porosity, density, and/or void (pore) size throughout, or to comprise at least one of pore size, porosity, and/or density being varied within the structure. For example, an open porous metal structure may have a different pore size and/or porosity at different regions, layers, and surfaces of the structure. The ability to selectively tailor the structural properties of the open porous metal, for example, enables tailoring of the structure for distributing stress loads throughout the surrounding tissue and promoting specific tissue ingrown within the open porous metal.

In other embodiments, an open porous metal structure may comprise an open cell polyurethane foam substrate coated with Ti-6Al-4V alloy using a low temperature arc vapor deposition process. Ti-6Al-4V beads may then be sintered to the surface of the Ti-6Al-4V-coated polyurethane foam substrate. Additionally, another embodiment of an open porous metal structure may comprise a metal substrate combined with a Ti-6Al-4V powder and a ceramic material, which is sintered under heat and pressure. The ceramic particles may thereafter be removed leaving voids, or pores, in the substrate. An open porous metal structure may also comprise a Ti-6Al-4V powder which has been suspended in a liquid and infiltrated and coated on the surface of a polyurethane substrate. The Ti-6Al-4V coating may then be sintered to form a porous metal structure mimicking the polyurethane foam substrate. Further, another embodiment of an open porous metal structure may comprise a porous metal substrate having particles, comprising altered geometries, which are sintered to a plurality of outer layers of the metal substrate. Additionally, an open porous metal structure may be fabricated according to electron beam melting (EBM) and/or laser engineered net shaping (LENS). For example, with EBM, metallic layers (comprising one or more of the biomaterials, alloys, and substrates disclosed herein) may be coated (layer by layer) on an open cell substrate using an electron beam in a vacuum. Similarly, with LENS, metallic powder (such as a titanium powder, for example) may be deposited and coated on an open cell substrate by creating a molten pool (from a metallic powder) using a focused, high-powered laser beam.

Because various portions of the tibial implant 100 can be formed of a porous structure, like the above-described porous tantalum, these portions can promote bone ingrowth of the resected surfaces of the tibia surrounding the tibial implant 100.

Differently-sized tibial implants can be made available to the surgeon or other user which are similar to the previously described tibial implant 100 and can increase/decrease in size according to standard sizes of tibial implants. The tibial implant can have a reduced width/length/height or an increased width/length/height, based, in part, on an overall size of the patient's tibia and the dimensions of the remaining portions of the tibia. Additionally, the use of a symmetric tibial implant as described herein can be configured for use in various procedures including a TKA procedure, TKA sparing ACL/PCL, and bicompartmental as well as the unicomparmental procedure as described above.

A method of performing a knee arthroplasty for knee replacement (e.g., a unicompartmental knee replacement) is also disclosed according to an example of the present disclosure. The method can include resecting a proximal surface of a tibia and attaching a symmetric tibial implant configured to be disposed in a left tibia medial compartment, a left tibia lateral compartment, a right tibia medial compartment, or a right tibia lateral compartment to the resected proximal surface of the tibia. According to some examples, the method can further include attaching a fixation component to one of two apertures formed in a baseplate of the tibial implant. A first of the two apertures can be formed in a posterior portion of the baseplate and the other of the two apertures can be formed in an anterior portion of the baseplate. In other examples, resecting a proximal surface of the tibia comprises resecting one or both of the medial and lateral articular surfaces of the tibia. According to other examples, resecting the proximal surface of the tibia can comprise resecting one or both of the medial and lateral articular surfaces of the left tibia, and resecting one or both of the medial and lateral articular surfaces of the right tibia.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may he used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A tibial implant configured for attachment to a tibia in a knee arthroplasty, the tibial implant comprising:
a baseplate having a first portion and a second portion, the first portion is symmetrically shaped, sized, and positioned with respect to the second portion; and
one or more fixation features configured to extend from a distal surface of the baseplate and configured for attachment to a resected proximal surface of the tibia.

2. The tibial implant of claim 1, wherein the baseplate is adapted to be disposed in a single compartment of the tibia and the tibia comprises either a right tibia or a left tibia.

3. The tibial implant of claim 2, wherein the single compartment comprises a left tibia medial compartment, a left tibia lateral compartment, a tight tibia medial compartment, or a right tibia lateral compartment.

4. The tibial implant of claim 3, wherein the first portion is more anteriorly disposed with respect to the second portion in the left medial compartment and the first portion is more posteriorly disposed with respect to the second portion in the left lateral compartment.

5. The tibial implant of claim 1, wherein the first portion comprises one half of the baseplate and the second portion comprises a second half of the baseplate.

6. The tibial implant of claim 1, wherein the one or more fixation features comprise:
a single integrally formed component generally centrally disposed between the first portion and the second portion; and
a removable component configured to be attached to one of at least two apertures formed in the baseplate, wherein a first one of the apertures is formed in the first portion and a second one of the apertures is formed in the second portion.

7. The tibial implant of claim 6, wherein the removable component is attached to a more anteriorly disposed of the at least two apertures.

8. The tibial implant of claim 1, wherein the baseplate has a first groove along an edge of the first portion and a matching second groove along an edge of the second portion.

9. The tibial implant of claim 1, wherein the tibial implant includes a porous structure,

10. The tibial implant of claim 9, wherein the porous structure includes tantalum.

11. The tibial implant of claim 9, wherein the porous structure comprises at least one of the distal surface of the baseplate and the one or more fixation features.

12. The tibial implant of claim 1, wherein the knee arthroplasty comprises a bicompartmental knee arthroplasty and the tibial implant comprises two tibial implants as recited in claim 1, a first of the two tibial implants is configured to be disposed in a medial compartment of the tibia and a second of the two tibial implants is configured to be disposed in a lateral compartment of the same tibia.

13. A unicompartmental tibial component comprising:
a baseplate having a first portion and a second portion, the first portion is symmetrically shaped and sized with respect to the second portion such that the tibial component is configured to be disposed in a left tibia medial compartment, a left tibia lateral compartment, a right tibia medial compartment, or a right tibia lateral compartment; and
one or more fixation features configured to be attached to and extending from a distal surface of the baseplate and configured for attachment to a resected proximal surface of a tibia.

14. The tibial component of claim 13, wherein an orientation of the baseplate is reversible such that a posterior edge of the baseplate becomes an anterior edge of the baseplate.

15. The tibial component of claim 14, wherein the one or more fixation features comprise:
a single integrally formed peg generally centrally disposed between the first portion and the second portion; and
a removable peg configured to be attached to one of at least two apertures formed in the baseplate, wherein a first one of the apertures is formed in the first portion and a second one of the apertures is formed in the second portion.

16. The tibial component of claim 15, wherein the removable peg is attached to the anteriorly disposed of the at least two apertures.

17. A method of performing a knee arthroplasty, the method comprising:
resecting a proximal surface of a tibia; and
attaching a symmetric tibial implant configured to be disposed in a left tibia medial compartment, a left tibia lateral compartment, a right tibia medial compartment, or a right tibia lateral compartment to the resected proximal surface of the tibia.

18. The method of claim 17, comprising attaching a fixation component to one of at least two apertures formed in a baseplate of the tibial implant, wherein a first one of the apertures is formed in a posterior portion of the baseplate and a second one of the apertures is formed in an anterior portion of the baseplate.

19. The method of claim 17, wherein resecting a proximal surface of the tibia comprises resecting one or both of the medial and lateral articular surfaces of the tibia.

20. The method of claim 17, wherein resecting a proximal surface of the tibia comprises:
resecting one or both of the medial and lateral articular surfaces of the left tibia; and
resecting one or both of the medial and lateral articular surfaces of the right tibia.
